(19) United States
(12) Patent Application Publication
(10) Pub. No.: US 2021/0370005 A1
(43) Pub. Date: Dec. 2, 2021

(54) ORO-NASAL PATIENT INTERFACE FOR TREATING SLEEP DISORDERED BREATHING

(71) Applicant: BESPOKE MEDICAL INNOVATIONS PTY LTD, Castle Hill (AU)

(72) Inventors: Christopher DODDS, Castle Hill (AU); John HILTON, Castle Hill (AU); Philip KWOK, Castle Hill (AU)

(73) Assignee: Bespoke Medical Innovations Pty Ltd, Castle Hill (AU)

(21) Appl. No.: 16/975,306

(22) PCT Filed: Feb. 18, 2019
(86) PCT No.: PCT/AU2019/050132

§ 371 Date: Aug. 24, 2020

(30) Foreign Application Priority Data
Feb. 23, 2018 (AU) 2018900585

(51) Int. Cl. A61M 16/06; B33Y 80/00; A61M 16/08

(52) U.S. Cl. CPC A61M 16/0683; B33Y 80/00; A61M 16/0666; A61M 2207/00; A61M 16/0825; A61M 2016/0661; A61M 16/0622

(57) ABSTRACT

A patient interface (200, 300) comprising: a seal-forming structure (210, 220, 310, 320) including a face-engaging surface that is personalised to the patient's facial contour and to form a seal with the patient's face at least on or below the bridge of the patient's nose and between the patient's lower lip and on or above the patient's chin, preferably proximal to the patient's mentolabial crease, the face-engaging surface including at least one nare port (211, 311) shaped and sized to align with the patient's nares; an anterior surface (205, 305) including a connection port; wherein the seal-forming structure (210, 220, 310, 320) is non-deformable in response to headgear tension or pressurised air received within the seal-forming structure (210, 220, 310, 320).

### FIELD OF THE INVENTION

The present invention relates to an oro-nasal patient interface for treating sleep disordered breathing (SDB).

### BACKGROUND TO THE INVENTION

Sleep apnoea is a form of SDB and is commonly treated with equipment providing continuous positive airway pressure (CPAP), typically between 4 and 20 cm H₂O air pressure, to the nasal passage of the patient via a patient interface that forms a seal against the patient's face. The air pressure may be higher than 20 cm H₂O for bi-level positive airway pressure. CPAP acts as a pneumatic splint and may prevent upper airway occlusion by pushing the soft palate and tongue forward and away from the posterior oropharyngeal wall.

The application of a supply of air at positive pressure to the entrance of the airways of a patient is facilitated by the use of a patient interface, such as a nasal mask, full-face mask or nasal pillows. A range of patient interface devices with deformable seal-forming structures made from silicone, gel and foam suffer from being one or more of obtrusive, aesthetically undesirable, poorly fitting, difficult to use and uncomfortable especially when worn for long periods of time or when a patient is unfamiliar with a system. Masks designed solely for aviators, as part of personal protection equipment or for the administration of anaesthetics may be tolerable for their original application, but nevertheless be undesirably uncomfortable to be worn for extended periods, for example, while sleeping.

For a patient interface where the seal-forming structure is a flexible mask cushion, the combined push vector can be calculated as the largest pressurized air cross section multiplied by the air pressure plus the forces transmitted through the flexible mask cushion walls. Prior masks with a flexible and deformable mask cushion have used increased air pressure to produce a stronger push onto a stiffer peripheral wall portion or rim of the flexible mask cushion to provide a stronger sealing force with higher air pressures. One drawback is that such a flexible mask cushion is bulky, is not visually aesthetic and obstructs a relatively large portion of the patient's face. Alternate prior masks have large skin contact areas and/or require a high level of headgear tension for their mask straps and therefore very uncomfortable for patients because CPAP therapy is typically required for prolonged duration, at least 5 hours each night.

Prior masks with a flexible and deformable mask cushion require headgear straps to be highly tensioned until a perimeter seal is achieved by the mask cushion. Areas of interference are further compressed. These prior masks attempt to make these areas of interference flex and stretch more to accommodate population variation of facial anthropometric differences. A rigid seal-forming structure results in minimal or nominal concentrated facial compression because the seal-forming structure is personalised or customised for an individual patient without high or low gaps between the seal-forming structure and the patient's face. Therefore headgear tension used for a rigid seal-forming structure can be lower relative to prior masks with deformable or soft mask cushions, resulting in high levels of comfort without facial marking (red marks) for the patient.

Traditional oro-nasal masks include full face masks that seal around the patient's nose and mouth. Due to their size and bulk, they may be less comfortable and more intrusive than other masks due to physiological reasons including claustrophobia or clithrophobia. Oro-nasal masks are typically bulky and heavy and can interfere with patient comfort and prevent wearing of eyeglasses.

Patient interfaces typically include a seal-forming portion or seal-forming structure. One traditional type of seal-forming portion extends around the periphery of the patient interface, and is intended to seal against the user's face when force is applied to the patient interface with the seal-forming portion in confronting engagement with the user's face. Traditional seal-forming portions may consist of an air or fluid filled cushion, or a moulded or formed surface of a resilient seal element made of an elastomer such as a rubber. With this type of seal-forming portion, if the fit is not adequate, there will be gaps between the seal-forming portion and the face, and additional force will be required to force the patient interface against the face in order to achieve a seal. This may lead to pressure sores or marking of the patient's skin from localised or concentrated forces acting upon specific parts of the patient's face, especially at the non-fleshy areas of the patient's face.

### SUMMARY OF THE INVENTION

The inventive concept arises from a recognition that a rigid seal-forming structure can be comfortable for a patient.

The present invention, in one aspect, comprises a patient interface for sealed delivery of a flow of air at a continuously positive pressure with respect to ambient air pressure to an entrance to a patient's airways including at least a patient's nares and mouth. The patient interface is configured to maintain a therapy pressure in a range of about 4 cm H2O to about 30 cm H2O above ambient air pressure in use, throughout the patient's respiratory cycle, while the patient is sleeping, to ameliorate sleep disordered breathing. The patient interface comprises a seal-forming structure including a face-engaging surface that is personalised to the patient's facial contour and to form a seal with the patient's face at least on or below the bridge of the patient's nose and between the patient's lower lip and on or above the patient's chin, preferably proximal to the patient's mentolabial crease, the face-engaging surface including at least one nare port shaped and sized to align with the patient's nares. The patient interface also comprises an anterior surface including a connection port. The seal-forming structure is non-deformable in response to headgear tension or pressurised air received within the seal-forming structure.

The face-engaging surface may be personalised via a digitising process.

The face-engaging surface may further comprise at least one mouth aperture positioned and shaped to align with the patient's mouth in a relaxed state.

The at least one mouth aperture may be a plurality of mouth apertures spaced apart across the distance of the width of the patient's mouth, the size and location of the plurality of mouth apertures are personalised to the patient's mouth breathing.

The spaced apart mouth apertures may have a variable size ranging from larger centrally located apertures to smaller peripheral apertures.

The spaced apart mouth apertures may be in the shape of circular holes or elongate horizontal slots.

The patient interface may further comprise a plenum chamber formed by at least the face-engaging surface and the anterior surface.

The face-engaging surface may have an upper lip contact region to contact or form a seal with the patient's upper lip to form a nasal chamber and a mouth chamber.

The patient interface may further comprise upper and lower headgear connection arms extending from the anterior surface.

The distal ends of the upper and lower headgear connection arms may comprise elongate slots to receive upper and lower headgear straps of a positioning and stabilising structure, respectively.

The elongate slots may comprise a jagged sawtooth edge configured to grip the headgear straps.

The upper headgear connection arms may be personalised to a patient's facial contour and are shaped to direct an upper headgear tension vector of the upper headgear straps in direction between the patient's eyes and ears.

The lower headgear connection arms may be personalised to a patient's facial contour and are shaped to direct a lower headgear tension vector of the lower headgear straps in a direction substantially parallel to the Frankfort horizontal direction and avoid extending across the patient's ears.

The connection port may be positioned in front of the at least one mouth aperture.

The connection port may be configured to releasably connect with a rotatable vented elbow.

The face-engaging surface may comprise a roughened surface portion proximal to the patient's mouth.

The present invention, in another aspect, comprises a method for manufacturing a patient interface for treating sleep disordered breathing at a pressure elevated above atmospheric pressure in a range of at least 4 cm H2O. The method comprises personalising a face-engaging surface to a patient's facial contour and to form a seal with the patient's face at least on or below the bridge of the patient's nose and between the patient's lower lip and on or above the patient's chin, preferably proximal to the patient's mentolabial crease, the face-engaging surface including at least one nare port shaped and sized to align with the patient's nares. The seal-forming structure is made from a non-deformable material that is non-deformable in response to headgear tension or pressurised air received within the seal-forming structure.

The method may further comprise initial steps of: capturing images of the patient's face and generating a three-dimensional (3D) model using the captured images.

The patient interface may be manufactured using an additive manufacturing process, for example, 3D printing.

The method may further comprise initial steps of: capturing images of the patient's face and generating a three-dimensional (3D) model using the captured images.

The seal-forming structure may be manufactured using an additive manufacturing process.

Rigid in the context of the present invention means non-deformable in response to headgear tension or pressurised air received within the seal-forming structure. In a preferred form, rigidity of seal-forming structure for a patient interface for treating sleep apnea is measured in terms of deformation the seal-forming structure under a typical treatment condition that is simulated. The patient interface is rest against a simulated patient's face with loosened headgear straps and no air flow or treatment pressure. A first distance is measured between the plane of the alar nasal sulci and the distal end of the skin contacting surface of the seal-forming structure of the patient interface to the nearest 0.5 mm. The patient interface is then connected to a flow generator and a treatment pressure of 20 cm H2O air pressure is applied. The headgear straps are tightened such that there are no detectable leaks between the patient interface and the patient's face. A second distance is measured between the plane of the alar nasal sulci and the distal end of the skin contacting surface of the seal-forming structure of the patient interface to the nearest 0.5 mm. Rigid preferably means that there is no difference between the first and second distance measurements.

The state of the art full face patient interface is the ResMed™ AirTouch™ F20 full face mask (released in May 2017) which is compared to the present invention.

| | Mask weight without elbow or | | |
|---|---|---|---|
| | headgear straps | | |
| | Frame | Cushion | Total |
| Mask | Module/Chassis | Module | weight |
| ResMed ™ AirFit ™ | 33.8 grams | 38.4 grams | 73.2 grams |
| F20 (medium size) | | | |
| Present Invention | | | 20.2 grams |

Referring to the comparative table above, the present invention is significantly lighter than a medium sized ResMed AirTouch F20 full face mask by about 53 grams. The present invention is about 27% the weight of the medium sized ResMed™ AirTouch™ F20 full face mask. Also, the present invention is approximately ⅓ the volume of the medium sized ResMed AirTouch F20 full face mask making it less bulky, less claustrophobic and more visually appealing. Also the present invention has less internal deadspace that may advantageously improve CO2 washout performance of the patient interface.

Other advantages and features according to the invention will be apparent to those of ordinary skill upon reading this application.

### BRIEF DESCRIPTION OF THE DRAWINGS

Embodiments of the invention will be described with respect to the figures, in which like reference numbers denote like elements and in which:

FIG. 1 is a photo of a prior art full face mask compared against a first embodiment of an oro-nasal mask according to the present invention and a second embodiment of an oro-nasal mask according to the present invention;

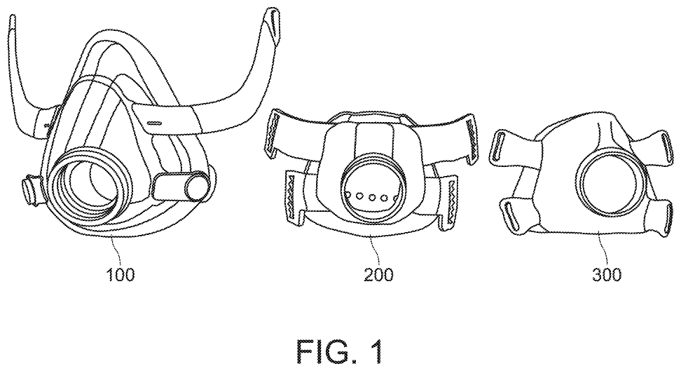

FIG. 2 is a photo of a front view of prior art full face mask worn by a patient depicting the geometric footprint of the prior art full face mask;

FIG. 3 is a photo of an angled side view of the prior art full face mask worn by a patient;

FIG. 4 is a photo of another angled side view of the prior art full face mask worn by a patient;

FIG. 5 is a photo of a side view of the prior art full face mask worn by a patient depicting the depth of the prior art full face mask;

FIG. 6 is a photo of a perspective view of the first embodiment of an oro-nasal mask according to the present invention with headgear straps and a vented swivel elbow attached;

FIG. 7 is a photo of a rear view of the first embodiment of an oro-nasal mask according to the present invention with headgear straps;

FIG. 8 is a photo of an angled side view of the first embodiment of an oro-nasal mask according to the present invention worn by a patient;

FIG. 9 is a photo of a front view of the first embodiment of an oro-nasal mask according to the present invention worn by a patient;

FIG. 10 is a photo of a perspective view of the second embodiment of an oro-nasal mask according to the present invention with headgear straps and a vented swivel elbow attached

FIG. 11 is a photo of a rear view of the second embodiment of an oro-nasal mask according to the present invention with headgear straps;

FIG. 12 is a photo of a front view of the second embodiment of an oro-nasal mask according to the present invention worn by a patient;

FIG. 13 is a photo of an angled side view of the second embodiment of an oro-nasal mask according to the present invention worn by a patient;

FIG. 14 is a photo of a zoomed in angled side view of the second embodiment of an oro-nasal mask according to the present invention worn by a patient;

### DETAILED DESCRIPTION OF THE INVENTION

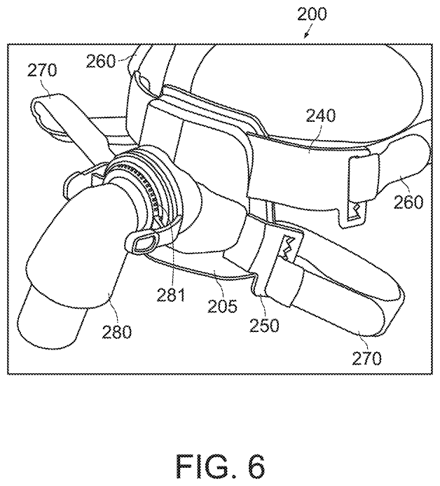

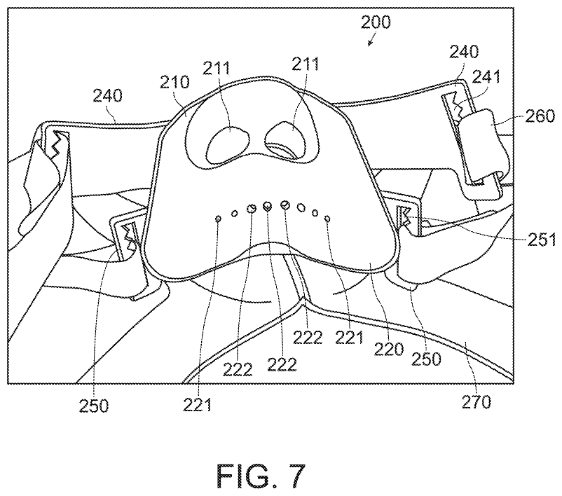

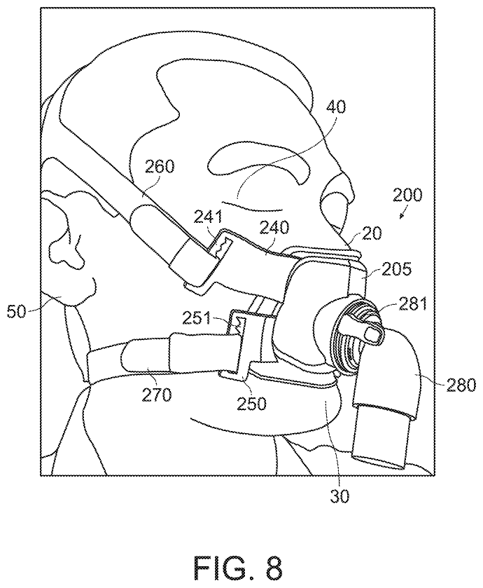

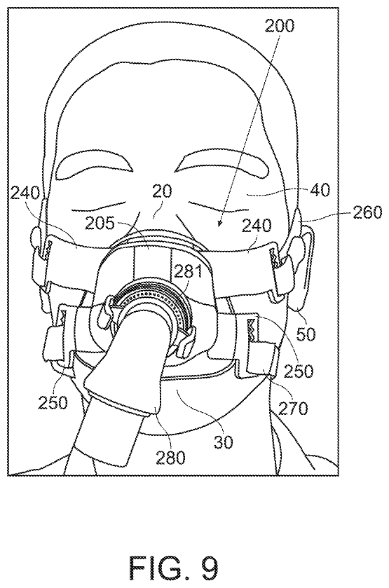

Referring to FIGS. 6 to 9, a first embodiment of patient interface 200 for sealed delivery of a flow of air at a continuously positive pressure with respect to ambient air pressure to an entrance to a patient's airways including at least a patient's nares and mouth is provided. The patient interface 200 is configured to maintain a therapy pressure in a range of about 4 cm H2O to about 30 cm H2O above ambient air pressure in use, throughout the patient's respiratory cycle, while the patient is sleeping, to ameliorate sleep disordered breathing, The patient interface 200 comprises a seal-forming structure including a face-engaging surface that is personalised to the patient's facial contour. The seal-forming structure forms a seal with the patient's face at least on or below the bridge 20 of the patient's nose and between the patient's lower lip and on or above the patient's chin. Preferably, the seal-form structure seals proximal to the patient's mentolabial crease 30. The face-engaging surface includes at least one nare port 211 shaped and sized to align with the patient's nares. Preferably, a pair of nare ports 211 is provided where each nare port 211 is separated from the other by a columella relief portion. The patient interface 200 also comprises an anterior surface 205 including a connection port. The seal-forming structure is non-deformable in response to headgear tension or pressurised air received within the seal-forming structure. For example, the seal-forming structure and the patient interface may be 3D printed using a rigid biocompatible material such as acrylonitrile butadiene styrene (ABS) plastic or polycarbonate plastic.

The seal-forming structure 210, 220 is part of the patient interface 200 and in use is arranged to surround an entrance to the airways of the patient so as to facilitate the supply of air at positive pressure to the airways. The seal-forming structure 210, 220 extends in use about the entire perimeter of a plenum chamber. The plenum chamber is a portion of the patient interface 200 having walls enclosing a volume of space which has air therein pressurised above atmospheric pressure in use. The plenum chamber may be formed by at least the face-engaging surface and the anterior surface 205. The plenum chamber is preferably rigid and not readily deformable to finger pressure (or from pressurised air within the plenum chamber 92). In other embodiments, the seal-forming structure and face-engage surface is rigid, but the plenum chamber and/or anterior surface 205 are semi-rigid or non-rigid in response to finger pressure which may cause the patient to perceive the patient interface 200 as less medical in appearance or more comfortable. The patient interface 200 also comprises headgear 250, 260 to retain the patient interface 200 against the patient's face during therapy. The headgear 250, 260 functions as a positioning and stabilising structure for use on a patient's head. The headgear 250, 260 may comprise ties (e.g. formed of soft flexible elastic material) and stiffeners (to limit flexibility in certain directions or elongation in certain directions). The patient interface 200 has or is operatively connectable to an air conduit to deliver air at positive pressure from a positive airway pressure (PAP) device (not shown). The patient interface 200 may comprise at least one vent or be operably connectable to an elbow 280 with a vent 281 to enable an intentional flow of air from an interior of the patient interface 200 to ambient air for the purpose of allowing washout of exhaled gases by the patient.

The face-engaging surface is personalised via a digitising process. Preferably, the process is contactless without requiring any contact or deformation of the patient's face. Advantageously, this results in a more accurate and stable seal because the patient's facial contour is captured in its natural or relaxed state.

The face-engaging surface further comprises at least one mouth aperture 221, 222 positioned and shaped to align with the patient's mouth in a relaxed state. The at least one mouth aperture is a plurality of mouth apertures 221, 222 spaced apart across the distance of the width of the patient's mouth. The size and location of the plurality of mouth apertures 221, 222 are personalised to the patient's mouth breathing. The spaced apart mouth apertures have a variable size ranging from larger centrally located apertures 222 to smaller peripheral apertures 221.

The spaced apart mouth apertures are in the shape of circular holes 221, 222 or elongate horizontal slots 321, 322.

The face-engaging surface preferably has an upper lip contact region to contact or form a seal with the patient's upper lip to form a nasal chamber and a mouth chamber. The nasal chamber and mouth chamber may be fluidly connected to each other by intentionally designed air paths between these two chambers. The upper lip contact region is located below the nare ports 211 and above the mouth apertures 221, 222. In other embodiments, an upper lip contact region is not provided and therefore a single oro-nasal chamber is formed.

Upper and lower headgear connection arms 240, 250 extend from the anterior surface 205. Preferably the headgear connection arms 240, 250 are integrally formed with the patient interface 200. The distal ends of the upper and lower headgear connection arms 240, 250 comprise elongate slots 241, 251 to receive upper and lower headgear straps 260, 270 of a positioning and stabilising structure, respectively. The elongate slots 241, 251 comprise a jagged sawtooth edge configured to grip the headgear straps 260, 270. This provides resistance during use to limit relative movement of the headgear straps 260, 270 to the headgear connection arms 240, 250 to enable a comfortable and stable seal.

The upper headgear connection arms 240 are also personalised to a patient's facial contour. The upper headgear connection arms 240 are shaped to direct an upper headgear tension vector of the upper headgear straps 260 in direction between the patient's eyes 40 and ears 50. The upper headgear connection arms 240 may be curved and extend across the patient's face to a point corresponding to the greatest width of the cheek area of a patient's face such that upper headgear straps 260 can be easily tightened and rest on the patient's skin rather than slightly wrapping around the patient's face. Preferably, the upper headgear connection arms 240 are slightly spaced away from the patient's face when the patient interface 200 is donned and rest slightly against the patient's skin when the upper headgear straps 260 are tightened during therapy.

The lower headgear connection arms 250 are also personalised to a patient's facial contour. The lower headgear connection arms 250 are shaped to direct a lower headgear tension vector of the lower headgear straps in a direction substantially parallel to the Frankfort horizontal direction and avoid extending across the patient's ears. The lower headgear connection arms 250 may extend across the patient's face to a point corresponding to the greatest width of the jaw area of patient's face such that lower headgear straps 270 can be easily tightened and rest on the patient's skin rather than slightly wrapping around the patient's face. Preferably, the lower headgear connection arms 250 are slightly spaced away from the patient's face when the patient interface 200 is donned and rest slightly against the patient's skin when the lower headgear straps 250 are tightened during therapy

The connection port is positioned in front of the at least one mouth aperture. The connection port is configured to releasably connect with a rotatable vented elbow 280.

The face-engaging surface comprises a roughened surface portion proximal to the patient's mouth and the mouth apertures 221, 222. The roughened or textured surface permits some air flow between the face-engaging surface in this area and the patient's skin around the mouth to avoid build up of condensate. Advantageously this improves patient comfort because the face-engaging surface will feel less damp and clammy. Other surface finishing or coatings may be applied to enhance patient comfort. For example, the patient interface 200 can be less ‘sweaty’ and uncomfortable by creating a fine matt finish rather than smooth glossy finish at areas of the seal-forming structure that are brought into contact around the lip and chin area. The location and extent of the finishing may be controlled to ensure a stable seal without compromising treatment pressure is maintained while enhancing patient comfort. The roughened surface portion provides an intentionally designed micro leak to improve comfort without compromising treatment pressure.

Increased Patient Comfort with Mouth and Lip Sealing Method

The patient interface 200 supports the patient's lower lip to the more stationary top lip using minimal force to close the lips together to prevent mouth leaks. The patient interface 200 is suitable for patients whose mouths droop open or lips blow open under therapeutic pressure.

There is a small bump feature on the patient interface 200 that aligns with the furrow or mentolabial crease 30 on the patient's face which is below the lower lip and above the chin. This assists in supporting the patient's lower lip to remain upwards. This is possible because an image of the patient's face is captured with their mouth closed. In other embodiments, the seal-forming structure aligns with the patient's chin.

It is observed that the patient's face, in response to air pressure, distends outwards when the patient's face relaxes during sleep. When this occurs against a customised rigid seal-forming structure, the seal is further enhanced.

Controlled and Personalised Mouth to Nose Proportionate Breathing for Patient

Some clinical literature support the view that nasal administered CPAP is more effective than mouth administered CPAP therapy. Therefore an oro-nasal mask that provides a more effective clinical outcome is one which encourages the patient to breathe through their nose only. However, some patients need to breathe a small amount through their mouth or simply need to sigh and take a deep breath.

The patient interface 200 can maximally encourage mouth breathing whilst providing any variable or selectable level of mouth breathing desired. For example, a physician may request their patient only be allowed minimal mouth breathing and predominant nasal breathing. The patient interface 200 can be customised with enlarged or decreased mouth breathing orifice and may also modify the nasal opening cross sectional area if required (currently same size and naris opening). One example of increasing the ability to mouth breathe is because the patient has nasal resistance, or deviated septum.

The patient interface 200 can also vary the level size of mouth orifices from centre to outer ends of the mouth for comfort. For example, excessive air flow at the ends of mouth can be irritating to some patients.

It may also be desirable to encourage nasal breathing by throttling down mouth breathing through the provision of a micro diffuse leak in the region of the mouth to improve comfort. This is particularly useful for patients that reside in hot and humid climates because it prevents the skin becoming clammy.

The mouth orifices 221, 222 may be customised in two ways. Firstly, as the patient's lips open to inhale, the centre of the mouth typically opens further than the distal ends of the mouth. Therefore the size of orifices 221, 222 may be larger at the centre than ends of the mouth. Secondly, the contour of the lips coming together can also be considered during the customisation and manufacture of the patient interface 200 such that the patient does not need to open their mouth excessively or more than their natural resting state to communicate with the air supply.

Intimacy and Minimal Size According to Individual Patient

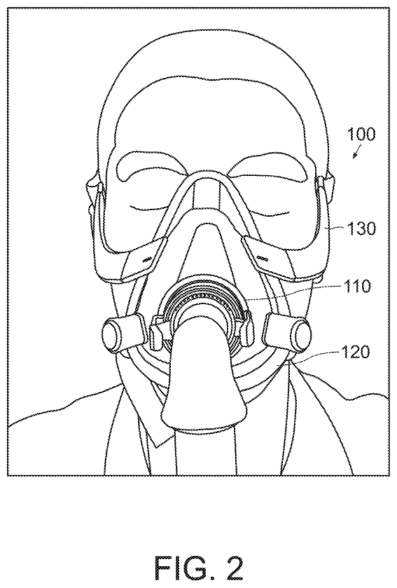

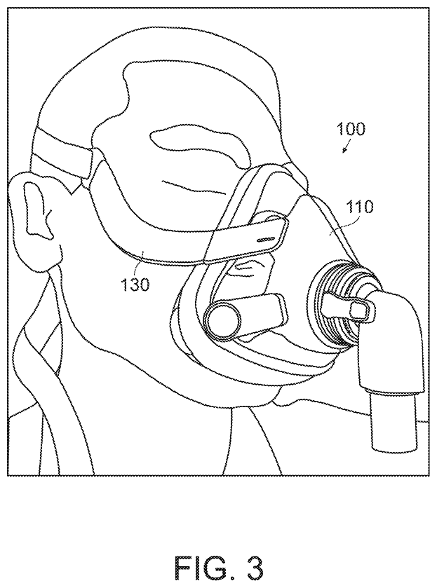

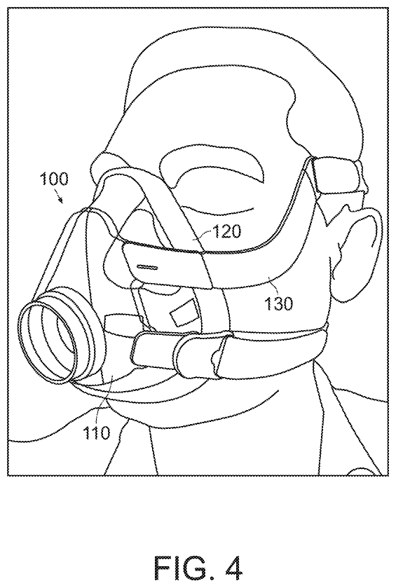

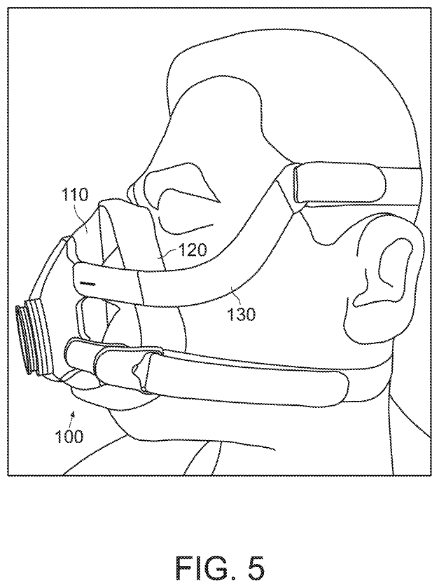

The patient interface 200 has a customised mask shell 205 that is an intimate form with the patient's face and therefore produces a very low profile with the patient's face. The customised patient interface 200 can be the lowest possible profile and size for a specific patient. Referring to FIGS. 2 to 5, conventional full face masks provide space within the mask frame (i.e. plenum chamber) to typically accommodate the 95^{th} percentile of patients' anthropometry. In other words, the mask is typically oversized for most patients which results in an excessive amount of wasted space, decrease in comfort, reduced field of view for the patient, and increasing destabilising forces caused by tube drag from an increase in the moment arm about the mask elbow thereby leveraging or pivoting the mask from the patient's face more easily. In contrast, the compact and low profile personalised patient interface 200 exposes a larger portion of the patient's face below their eyes, their cheeks and chin compared to a traditional full face mask.

As the vertical facial plane can be assessed during the customisation and manufacture of the patient interface 200, the angle for a rotatable elbow 280 for releasable connection with the connection port can be customised for the individual. Swivelling elbows for prior art masks incorporate a rocking inlet port (e.g. ball and socket joint) where the angle can move toward or away from the patient to better compromise whether the tubing passes too close to the body or angled so much from the body to create destabilising angles.

Superior Stability of Patient Interface During Therapy

The patient interface 200 primarily rests against the patient's face with light contact around the nose and also down the lateral sides of the patient's face. The portion the extends at and below the lower lip and onto the chin is largely matching the patients facial profile in the relaxed state rather than say in a position whereby the jaw or mandible is pushed backwards and more importantly is cantilevered as the mask shell is rigid. When the patient interface 200 is correctly positioned on the patient's face, there is very minimal force applied to the patient's mandible, and the forces are predominantly along the rigid/bony facial structures along the sides of the patient's nose.

By creating an intimate form, the patient can more readily practise prone body sleeping where patients lie on their body front with head turned. Since the patient interface 200 has a low profile on the patient's face, this increases the stability of the patient interface 200 which means it is more difficult to dislodge or destabilise from the patient's face during use where the patient may roll, turn and change position during sleep.

Image data to manufacture the patient interface 200 can use backwards compatible image data captured by a portable photogrammetry studio, for example, the applicant's portable photogrammetry studio disclosed in WO2016/187661 which is expressly incorporated by reference herein in its entirety. The portable photogrammetry studio may include a chin support that exposes the chin region and with less distortion such that the facial image data captured is backwards compatible to fit a patient interface for a patient at a later date if the patient subsequently finds that they require a full face mask instead of a nasal mask. This also means that the patient is not required to return to a scanning site to be rescanned and have a patient interface manufactured.

Improved Clinical Outcome Due to Optimised Anatomical Positioning of Patient'S Mandible/Jaw Bone

The patient interface 200 does not excessively press against the patient's mandible in contrast to prior full face masks. If lower headgear straps of prior full face masks are overtightened, the patient's jaw can be pushed backwards which is opposite to the intent of mandibular advancement/mouthguard devices for treating sleep apnea. The patient interface 200, in one embodiment, may reduce the Apnea-Hypopnea Index (AHI) by not closing the upper airways to the same extent as a conventional full-face mask and it is possible for treatment pressure to be reduced. Reducing treatment pressure may lead to improved patient compliance because there is less breathing discomfort. Also patient interfaces tend to perform better when not required to operate at high treatment pressures.

Offsets and comfort regions for the seal-forming structure may be provided around the nasal region, (for example, a personalised rigid seal-forming structure disclosed in the applicant's Australian provisional patent application 2017904973 which is expressly incorporated by reference herein in its entirety). The area from the patient's lower lip and below may have none or a minimal offset because this represents the natural position of the patient's mouth region. The seal is assisted by the mouth region distending towards the patient interface 200 to provide a seal, thus removing most of the backwards forces present in prior full face masks.

Improved Patient Comfort with Natural Jaw Position During Therapy

When the patient's face is relaxed, images of a comfortable mouth position for that patient can be captured to enable the design and manufacture of a customised mouth portion of the patient interface 200. For example, when the patient's face is relaxed with the lips together to avoid leak, a more comfortable and natural position has the jaw slightly open or teeth not clenched together. A portable photogrammetry studio can scan the patient's face in this natural position. It has been observed on a patient that it was more comfortable and natural to have the patient's jaw slightly open which tends to occur when the patient's face is a relaxed state during sleep. For example, during facial scanning, a series of discrete thickness bite blocks is provided to determine the most comfortable open jaw position for a patient which then allows the patient's lips in their natural/relaxed closed state to be scanned while the furrow or mentolabial crease 30 at the patient's lower jaw is comfortably positioned. The output of a final mask perimeter shape and customised mouth portion supports lip closure and provides the patient the freedom to have their jaw in the most comfortable position.

Freedom for Patient to Slightly Open Mouth Mitigating the Need for an Anti-Asphyxia Valve

In one embodiment, an anti-asphyxia valve (AAV) may not be required in contrast to traditional full face masks. AAVs are used to mitigate the risk of suffocation if the CPAP flow generator fails and the patient interface remains secured on the patient's face covering the patient's airways. One reason that the patient interface does not require an AAV is because the patient interface 200 does not press moderately against the patient's chin and provides the patient with the ability to open their mouth at will which can reduce anxiety having the mouth heavily closed over, or take a deep breath as desired, and without needing to reposition the mask by hand. The rigidity of the seal-forming structure of the patient interface means that the seal-forming structure does not deform and travel to resist the patient's movement. In contrast, traditional deformable seal-forming structures made from LSR or foam are designed to deform and thus the patient's is fighting to break free from the seal while the seal-forming structure is deforming as intended to maintain the seal.

However, with the mouth uninhibited, the patient interface 200 is inherently safer potentially avoiding the need for an AAV.

Customised Venting

A vent (not shown) may be constructed to be optimally located on the patient interface 200 considering fluid dynamics that occur within the patient interface 200 based on each specific patient. For example, the vent can be positioned in the most effective position relative to the connection port and the patient's nares and mouth to ensure optimal carbon dioxide washout. Conversely, if the patient's physician requires additional CO2 drive, the CO2 rebreathing may be increased. If CO2 washout is of lesser importance, the customised location provides a patient and bed partner with the ability to locate the vent direction based on typical sleeping positions to avoid air jetting disturbance.

Reduced Cost to Manufacture and Distribute

Compared to some prior full face masks which have a separate frame module and cushion module which releasably attach to each other, the patient interface 200 comprises the frame and cushion as a single component. Manufacturing cost is substantially reduced, assembly methods are not required and the quantity of material is reduced by not requiring connection components.

Improved Headgear Location and Strap Vectoring According to Facial Anatomy of Individual

The orientation of the headgear slots 241, 251 can be optimally positioned for a specific patient. The headgear connection arms 240, 250 and are intimate and follow the patient's cheek contour and are formed to be slightly held above the skin surface rather than contact and compress the skin surface. By forming the headgear connection arms 240, 250 such that there is a clearance with the patient's skin, this accounts for compression of soft facial tissue when tightening the heading straps 260, 270 to retain the patient interface 200 against the patient's face during use but prevents the seal-forming structure from pulling into the patient's face immediately on tightening. The headgear connection arms 240, 250 are close fitting while providing an optimised function.

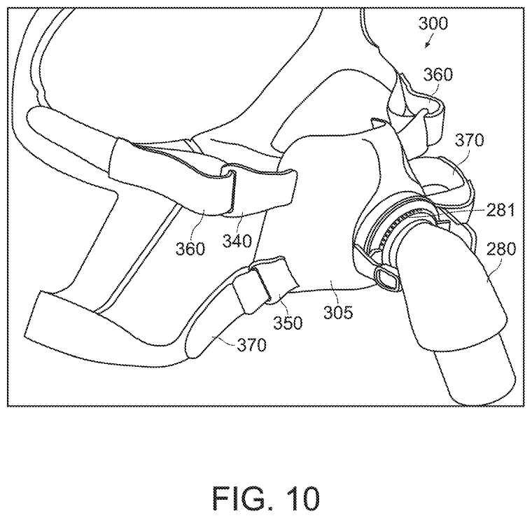

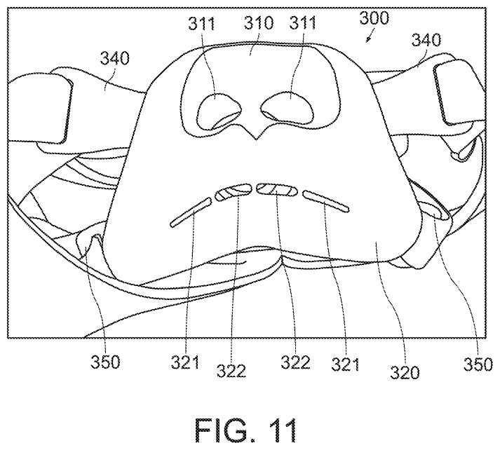

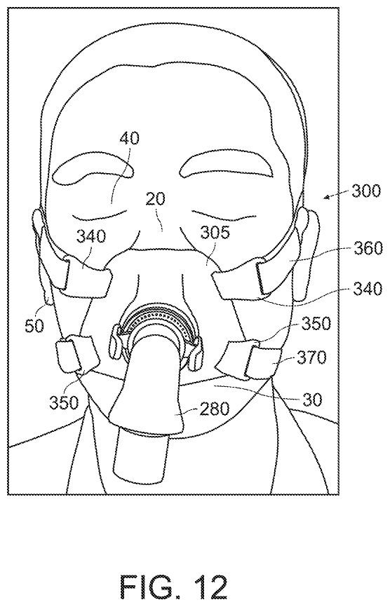

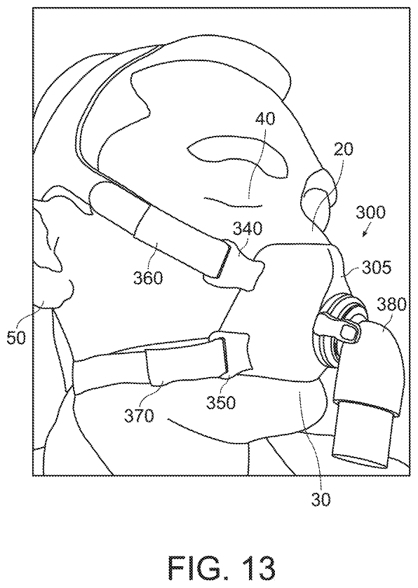

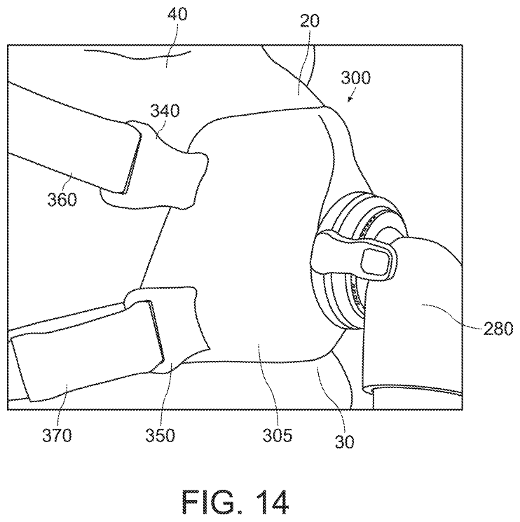

Referring to FIGS. 10 to 14, a second embodiment of patient interface 300 for sealed delivery of a flow of air at a continuously positive pressure with respect to ambient air pressure to an entrance to a patient's airways including at least a patient's nares and mouth is provided. The second embodiment has a smaller facial footprint than the first embodiment and also has a lower profile. One observable difference is that the headgear connection arms 340, 350 of the second embodiment are shorter and smaller than the headgear connection arms 240, 250 of the first embodiment. The anterior surface 305 of the second embodiment is less angular and more smooth than the anterior surface 205 of the first embodiment.

When a particular material is identified as being preferably used to construct a component, obvious alternative materials with similar properties may be used as a substitute. Furthermore, unless specified to the contrary, any and all components herein described are understood to be capable of being manufactured and, as such, may be manufactured together or separately.

Moreover, in interpreting the disclosure, all terms should be interpreted in the broadest reasonable manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements.

It is therefore to be understood that numerous modifications may be made to the illustrative examples and that other arrangements may be devised without departing from the spirit and scope of the technology.

1. An oro-nasal patient interface for sealed delivery of a flow of air at a continuously positive pressure with respect to ambient air pressure to an entrance to a patient's airways including at least a patient's nares and mouth, wherein the patient interface is configured to maintain a therapy pressure in a range of about 4 cm H₂O to about 30 cm H₂O above ambient air pressure in use, throughout the patient's respiratory cycle, while the patient is sleeping, to ameliorate sleep disordered breathing, said patient interface comprising:
a seal-forming structure including a face-engaging surface that is personalised to the patient's facial contour and to form a seal with the patient's face at least on or below the bridge of the patient's nose and between the patient's lower lip and on or above the patient's chin, preferably proximal to the patient's mentolabial crease, the face-engaging surface including at least one nare port shaped and sized to align with the patient's nares;
an anterior surface including a connection port;
wherein the seal-forming structure is non-deformable in response to headgear tension or pressurised air received within the seal-forming structure; and
the seal-forming structure has a glass transition temperature 100 degrees C. and is formed of a non-photoreactive material.

2. The oro-nasal patient interface according to claim 1, wherein the face-engaging surface is personalised via a digitising process.

3. The oro-nasal patient interface according to claim 1, wherein the face-engaging surface further comprises at least one mouth aperture positioned and shaped to align with the patient's mouth in a relaxed state.

4. The oro-nasal patient interface according to claim 3, wherein the at least one mouth aperture is a plurality of mouth apertures spaced apart across the distance of the width of the patient's mouth, the size and location of the plurality of mouth apertures are personalised to the patient's mouth breathing.

5. The oro-nasal patient interface according to claim 4, wherein the spaced apart mouth apertures have a variable size ranging from larger centrally located apertures to smaller peripheral apertures.

6. The oro-nasal patient interface according to claim 5, wherein the spaced apart mouth apertures are in the shape of circular holes or elongate horizontal slots.

7. The oro-nasal patient interface according to claim 1, further comprising a plenum chamber formed by at least the face-engaging surface and the anterior surface.

8. The oro-nasal patient interface according to claim 1, wherein the face-engaging surface has an upper lip contact region to contact or form a seal with the patient's upper lip to form a nasal chamber and a mouth chamber.

9. The oro-nasal patient interface according to claim 1, further comprising upper and lower headgear connection arms extending from the anterior surface.

10. The oro-nasal patient interface according to claim 9, wherein the distal ends of the upper and lower headgear connection arms comprise elongate slots to receive upper and lower headgear straps of a positioning and stabilising structure, respectively.

11. The oro-nasal patient interface according to claim 10, wherein the elongate slots comprise a jagged sawtooth edge configured to grip the headgear straps.

12. The oro-nasal patient interface according to claim 9, wherein the upper headgear connection arms are personalised to a patient's facial contour and are shaped to direct an upper headgear tension vector of the upper headgear straps in direction between the patient's eyes and ears, the upper headgear connection arms being spaced apart from the patient's skin when the patient interface is donned and when the upper headgear straps are tightened, the upper headgear connection arms are brought into contact with the patient's skin.

13. The oro-nasal patient interface according to claim 9, wherein the lower headgear connection arms are personalised to a patient's facial contour and are shaped to direct a lower headgear tension vector of the lower headgear straps in a direction substantially parallel to the Frankfort horizontal direction and avoid extending across the patient's ears, the lower headgear connection arms being spaced apart from the patient's skin when the patient interface is donned and when the lower headgear straps are tightened, the lower headgear connection arms are brought into contact with the patient's skin.

14. The oro-nasal patient interface according to claim 3, wherein the connection port is positioned in front of the at least one mouth aperture.

15. The oro-nasal patient interface according to claim 1, wherein the connection port is configured to releasably connect with a rotatable vented elbow.

16. The oro-nasal patient interface according to claim 1, wherein the face-engaging surface comprises a roughened surface portion proximal to the patient's mouth.

17. A method for manufacturing an oro-nasal patient interface for treating sleep disordered breathing at a pressure elevated above atmospheric pressure in a range of at least 4 cm H₂O, the method comprising:
personalising a face-engaging surface to a patient's facial contour configured to form a seal with the patient's face at least on or below the bridge of the patient's nose and between the patient's lower lip and on or above the patient's chin, preferably proximal to the mentolabial crease, the face-engaging surface including at least one nare port shaped and sized to align with the patient's nares; and
wherein the seal-forming structure is made from a non-deformable material that is non-deformable in response to headgear tension or pressurised air received within the seal-forming structure; and
wherein the seal-forming structure has a glass transition temperature above 100 degrees C. and is formed of a non-photoreactive material.

18. The method according to claim 17, further comprising initial steps of:
capturing images of the patient's face; and
generating a three-dimensional (3D) model using the captured images.

19. The method according to claim 17, wherein the patient interface is manufactured using an additive manufacturing process
